(19) United States
(12) Patent Application Publication
(10) Pub. No.: US 2008/0161412 A1
(43) Pub. Date: Jul. 3, 2008

(54) Process for preparation of sertraline hydrochloride form I

(75) Inventors: Ronen Borochovitz, Ramat-Poleg (IL); Shalom Shabat, Yavne (IL)

Correspondence Address: KENYON & KENYON LLP, ONE BROADWAY, NEW YORK, NY, 10004, US

(73) Assignees: Teva Pharmaceutical Industries Ltd.; Teva Pharmaceuticals USA, Inc.

(21) Appl. No.: 12/009,185

(22) Filed: Jan. 16, 2008

Related U.S. Application Data
(63) Continuation of application No. 11/450,046, filed on Jun. 9, 2006, now abandoned.
(60) Provisional application No. 60/689,777, filed on Jun. 9, 2005.

(51) Int. Cl. A61K 31/136; C07C 209/84

(52) U.S. Cl. 514/657; 564/428

(57) ABSTRACT

Provided is a process for preparation of sertraline HCl Form I on an industrial scale.

### RELATED APPLICATIONS

The present application is a continuation of U.S. application Ser. No. 11/450,046, filed on Jun. 9, 2006, and claims the benefit of the U.S. Provisional Application No. 60/689,777 filed Jun. 9, 2005. The contents of which are incorporated herein by reference.

### FIELD OF INVENTION

The present invention encompasses a process for preparing Sertraline hydrochloride form I.

### BACKGROUND OF THE INVENTION

Sertraline hydrochloride, (1S-cis)-4-(3,4 dichlorophenyl)-1,2,3,4-tetrahydro-N-methyl-1-naphthalenamine hydrochloride having the formula

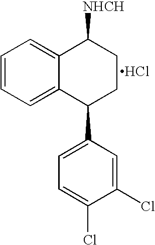

is approved, under the trademark ZOLOFT, by the U.S. Food and Drug Administration, for the treatment of depression, obsessive-compulsive disorder and panic disorder.

U.S. Pat. No. 5,248,699 (“the '699 patent”) discloses five crystalline forms of sertraline HCl, labeled Forms I, II, III, IV and V. It also provides a few examples for preparation of sertraline HCl Form I>

U.S. Pat. No. 6,872,853 and discloses U.S. publication No. 2004/0132828 discloses a process for preparing sertraline HCl form I by heating Sertraline HCl isopropanolate and by seeding a solution of Sertraline hydrochloride with form I.

The present invention relates to the solid state physical properties, i.e., polymorphism, of sertraline hydrochloride. These properties may be influenced by controlling the conditions under which sertraline hydrochloride is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account when developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid may have therapeutic consequences because it imposes an upper limit on the rate at which an orally-administered active ingredient may reach the bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC), and may be used to distinguish some polymorphic forms from others. A particular polymorphic form may also give rise to distinct properties that may be detectable by powder X-ray diffraction, solid state ¹³C NMR spectrometry and infrared spectrometry.

U.S. Pat. No. 5,248,699 reports that sertraline HCl Form I is thermodynamically stable. There is a need in the art to produce sertraline HCl Form I with processes suitable for industrial applicability.

### SUMMARY OF THE INVENTION

In one embodiment, the present invention provides a process for the preparation of sertraline HCl Form I comprising crystallizing sertraline HCl from n-butanol, wherein the crystallization comprises heating to a temperature of about 40° C. to about reflux temperature.

In another embodiment, the present invention provides a process for the preparation of sertraline HCl Form I comprising: combining sertraline base or sertraline salt with n-butanol and HCl at a temperature of about 20° C. to about 70° C. to obtain a precipitate; and crystallizing the precipitate from n-butanol, wherein the crystallization comprises heating to a temperature of about 40° C. to about reflux temperature.

In another embodiment, the present invention provides a process for the preparation of sertraline HCl Form I comprising the steps of:

a) heating sertraline HCl in n-butanol to a temperature of about 40° C. to about reflux temperature; and

b) crystallizing sertraline HCl.

### BRIEF DESCRIPTION OF THE FIGURE

FIG. 1 provides a powder XRD pattern of Form I.

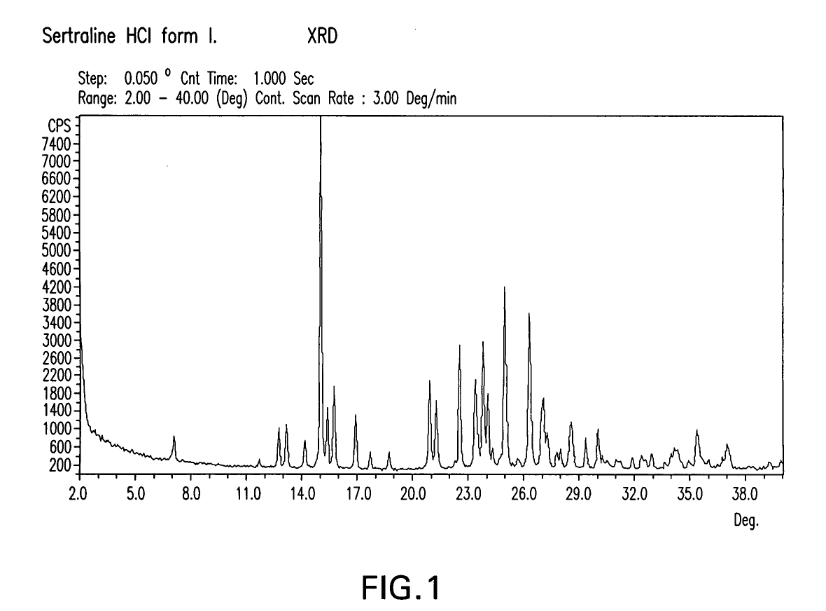

### DETAILED DESCRIPTION OF THE INVENTION

The present invention relates to a process for preparing sertraline HCl Form I by crystallizing it from n-butanol at a specific temperature range. The use of n-butanol allows for consistent production of Form I with relatively high polymorphic purity. Further, use of n-butanol allows the process to be carried out on an industrial scale without change of solvents. The use of n-butanol is preferred, in comparison to other solvents, because it can be used in relatively small amounts and consistently allows for production of Form I.

As used herein, the term “industrial scale” refers to a process that results in a batch of at least about 0.5 Kg. The batch is preferably at least about 1 Kg.

The present invention provides a process for the preparation of sertraline HCl Form I comprising crystallizing sertraline HCl from n-butanol, wherein the crystallization comprises heating to a temperature of about 40° C. to about reflux temperature.

The present invention also provides a process for the preparation of sertraline HCl Form I comprising: combining sertraline base or sertraline salt with n-butanol and HCl at a temperature of about 20° C. to about 70° C. to obtain a precipitate; and crystallizing the precipitate from n-butanol, wherein the crystallization comprises heating to a temperature of about 40° C. to about reflux temperature.

Sertraline salt may be converted to sertraline base by combining it with a water immiscible solvent, water and a base selected from the group consisting of: sodium hydroxide, potassium hydroxide and alkaline carbonate, to obtain a two phase system; separating the organic phase; and recovering. Preferably, the water immiscible solvent is a C₅ to C₁₂ hydrocarbon. Preferably, the C₅ to C₁₂ hydrocarbon is toluene. Preferably, the base is sodium or potassium hydroxide. Preferably, the two phase mixture is heated to a temperature of about 40° C. to about 90° C. The reaction results in sertraline in the organic phase. The organic phase may be recovered by washing it with water, concentrating it and removing the residual water immiscible solvent.

Preferably, the sertraline salt is sertraline mandelate.

Preferably, the HCl is HCl gas. The use of HCl in its gaseous form is more applicable on an industrial scale, with comparison to HCl solution. Preferably, the HCl is added until obtaining a pH of less than about 3, more preferably, a pH of less than about 1.

Optionally, the obtained precipitate is isolated prior to the crystallization.

Optionally, the precipitate is sertraline HCl Form II.

The crystallization from n-butanol comprises heating to a temperature of about 40° C. to about reflux temperature to obtain a solution. Preferably, the heating is done to a temperature of about 90° C. to about 100° C. The dissolution does not require changing the solvent, which increases the efficiency of the process. Preferably, the solution may be seeded with sertraline HCl Form I to induce crystallization. After the heating, the solution is further cooled, preferably, to a temperature of about 40° C. to about 0° C., more preferably, to a temperature of about 20° C. Preferably, the cooling is carried out from about 6 hours to about 48 hours, more preferably, about 12.

The resulting crystals may be recovered by conventional techniques such as filtration. The crystals may also be dried, under ambient or reduced pressure. The drying process may be accelerated by heating the crystals.

Optionally, the processes of the present invention may be applicable in industrial scale.

The present invention further encompasses a process for preparing a pharmaceutical formulation comprising combining the sertraline hydrochloride Form I made by the process of the present invention with at least one pharmaceutically acceptable excipient.

Pharmaceutical compositions of the present invention contain sertraline hydrochloride Form I. In addition to the active ingredient(s), the pharmaceutical compositions of the present invention may contain one or more excipients. Excipients are added to the composition for a variety of purposes.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants may be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which may cause the product to have pitting and other surface irregularities. A lubricant may be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate. Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, sertraline hydrochloride and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste. Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as guconic acid, lactic acid, citric acid or acetic acid, sodium gluconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill. In particular formulation challenges of direct compression tableting.

A capsule filling of the present invention may comprise any of the aforementioned blends and granulates that were described with reference to tableting, however, they are not subjected to a final tableting step.

Preferably, the pharmaceutical formulations of the present invention are solid dosage forms in the form of a tablet for the oral administration of sertraline hydrochloride. The highly pure sertraline hydrochloride used for preparing a tablet may be in the form of fine crystals. Preferably, the fine crystals have a particle size distribution such that 100% of the particles are below 200 microns, more preferably below 100 microns and most preferably below about 50 microns.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way.

### EXAMPLES

### Example 1

### Preparation of Sertraline Hydrochloride Form I in N-Butanol

### a) Preparation of Sertraline Base

To the slurry of sertraline mandelate (50 g) in toluene (250 ml) was added water (140 ml) and aqueous solution of 47% NaOH (37 ml). The reaction mixture was heated to 80° C. and stirred with a mechanical stirrer. After ½ an hour the organic phase was separated and water was added (67 ml), the organic phase was separated again and washed again with the same water quantity. After the second wash the organic solution was concentrated by distillation; then residual toluene was removed by repeated distillation with n-butanol. The product obtained is Sertraline base.

b) Preparation of Sertraline hydrochloride Form I

To the obtained residue of Sertraline base in n-butanol was added (180 ml) n-butanol and the mixture was heated to a temperature from 40° C. to 70° C. Then, charcoal SX1 was added, the solution was filtered and HCl gas was bubbled until pH 1 was reached. The obtained sertraline hydrochloride precipitate was then dissolved in the same reaction mixture followed by heating to 90-100° C. The solution was cooled during 12 hours to 20° C., followed by filtration of the solid, washing with n-butanol and drying. The dried solid (31 g) obtained was sertraline hydrochloride form I.

### Example 2

### Preparation of Sertraline Hydrochloride Form I in-Butanol by Seeding

Sertraline base was prepared according to the previous example.

Sertraline base obtained from 38 g Sertraline mandelate was dissolved in 180 ml of n-butanol. Active charcoal was added at a temperature of 40° C., and the mixture was stirred for 30 minutes and then filtered. Then, to the filtrate was bubbled HCl gas at a temperature of ˜45° C. until pH 1 was reached, followed by heating the slurry to 90-100° C. to obtain complete dissolution. After complete dissolution seeding of Sertraline hydrochloride form I was performed, followed by cooling to 20° C. for 12 hours. The obtained solid was filtered, washed with n-Butanol and dried. The dried solid obtained (20.85 g) was Sertraline hydrochloride form I.

### Example 3

### Process for the Preparation of Sertraline Hydrochloride form I on an Industrial Scale

A reactor was charged with water (90 kg), aqueous solution 47% of NaOH (44 kg), toluene (201 l) and Sertraline mandelate cryst. (40 kg). The reactor was heated to 70-80° C. and the mixture was stirred for 1 hour. The stirring was stopped and the phases were separated; the organic phase was washed twice with hot water (53 kg) at a temperature of 70-80° C. The separated organic solution was concentrated under vacuum. The residual toluene was completely replaced by repeated evaporation with n-butanol. To the obtained residue n-Butanol at a temperature of 40-45° C. was added (138 kg) active carbon SX1 (1 kg) and then, the slurry was filtered. The filtrate was washed with n-Butanol (34.4 kg) and HCl gas (˜6 kg) was bubbled into the solution maintaining the temperature in the range of 40-55° C. The reactor was then heated to 103-113° C. until complete dissolution and then, the solution was cooled to 85-90° C. and sertraline HCl form I (0.25 kg) was added for seeding. The mixture was cooled to 18-28° C. for about 12 hours. The solid was filtered, washed with n-Butanol and dried. The product was Sertraline hydrochloride form I (21.8 kg).

1. A process for the preparation of sertraline HCl Form I comprising the steps of:
a) heating sertraline HCl in n-butanol to a temperature of about 40° C. to about reflux temperature; and
b) crystallizing sertraline HCl.

2. The process of claim 1, wherein sertraline HCl is prepared by combining sertraline base or sertraline salt in n-butanol with HCl at a temperature of about 20° C. to about 70° C. to precipitate the sertraline HCl.

3. The process of claim 2, wherein the sertraline salt is sertraline mandelate.

4. The process of claim 2, wherein the HCl is added until obtaining a pH of less than about 3.

5. The process of claim 2, wherein the HCl is added until obtaining a pH of less than about 1.

6. The process of claim 2, wherein the precipitate is isolated prior to the crystallization.

7. The process of claim 2, wherein the precipitate is sertraline HCl Form II.

8. The process of claim 2, wherein the HCl is HCl gas.

9. The process of any of claims 1, wherein the solution is seeded with sertraline HCl Form I.

10. The process of claim 1, wherein the heating is done to a temperature of about 90° C. to about 100° C.

11. The process of any of claims 1, wherein after heating, the solution is further cooled to a temperature of about 40° C. to about 0° C.

12. The process of claim 11, wherein the solution is cooled to a temperature of about 20° C.

13. The process of claim 11, wherein the cooling is carried out from about 6 hours to about 48 hours.

14. The process of claim 1, wherein the resulting crystals are recovered.

15. The process of claim 14, wherein the crystals are further dried, under ambient or reduced pressure.

16. The process of claim 1, wherein the process is carried out on an industrial scale.

17. A process for preparing a pharmaceutical formulation comprising combining the sertraline hydrochloride Form I made by the process of claim 1, with at least one pharmaceutically acceptable excipient.
